(19) United States
(12) Patent Application Publication
(10) Pub. No.: US 2022/0175869 A1
(43) Pub. Date: Jun. 9, 2022

(54) FORMULATIONS INHIBITING SARS-CoV-2 VIRUS

(71) Applicant: Ankh Life Sciences Limited, Dublin 2 (IE)

(72) Inventors: Gene H. Zaid, Hutchinson, KS (US); Beth Ann Wolf, Hutchinson, KS (US); Robert Preston Moore, Great Bend, KS (US); Larry D. Crow, Hutchinson, KS (US); Jason H. West, Hutchinson, KS (US)

(73) Assignee: Ankh Life Sciences Limited, Dublin 2 (IE)

(21) Appl. No.: 17/540,810

(22) Filed: Dec. 2, 2021

Related U.S. Application Data
(60) Provisional application No. 63/121,767, filed on Dec. 4, 2020.

(51) Int. Cl. A61K 36/9066; A61K 36/888; A61K 36/185; A61K 31/375; A61K 31/12; A61K 36/8962; A61K 31/575; A61P 31/14

(52) U.S. Cl. CPC A61K 36/9066; A61K 36/888; A61K 36/185; A61P 31/14; A61K 31/12; A61K 36/8962; A61K 31/575; A61K 31/375

(57) ABSTRACT

The treatment of human subjects exposed to, or potentially exposed to SARS-CoV-2 virus, or suffering from COVID-19, comprises the step of administering to the human subjects a formulation comprising turmeric, Peganum harmala, and Arum palaestinum, and/or variants thereof. Methods of inhibiting SARS-CoV-2 viral replication are also described.

### CROSS-REFERENCE TO RELATED APPLICATIONS

The present application claims the priority benefit of U.S. Provisional Patent Application Ser. No. 63/121,767, filed Dec. 4, 2020, entitled FORMULATIONS INHIBITING SARS-CoV-2 VIRUS, incorporated by reference in its entirety herein.

### BACKGROUND OF THE INVENTION

### Field of the Invention

The present invention is broadly concerned with a new medicament for the inhibition of SARS-CoV-2 virus, the cause of the ongoing COVID-19 pandemic. More particularly, the invention is concerned with the use of formulations comprising turmeric, Peganum harmala, and Arum palaestinum, as effective agents inhibiting replication of the virus and the resulting COVID-19 disease.

### Description of the Prior Art

The COVID-19 pandemic, also known as the coronavirus pandemic, is an ongoing pandemic of coronavirus disease 2019 (COVID-19) caused by severe acute respiratory syndrome coronavirus 2 (SARS-CoV-2), first identified in December 2019 in Wuhan, China. COVID-19 mainly spreads through the air when people are near each other long enough, primarily via small droplets or aerosols, as an infected person breathes, coughs, sneezes, sings, or speaks. Transmission via fomites (contaminated surfaces) has not been conclusively demonstrated. It can spread as early as two days before infected persons show symptoms (presymptomatic), and from asymptomatic (no symptoms) individuals. People remain infectious for up to ten days in moderate cases, and two weeks or longer in severe cases.

Common symptoms include fever, cough, fatigue, breathing difficulties, and loss of smell and taste. Complications may include pneumonia and acute respiratory distress syndrome. The incubation period is typically around five days but may range from one to 14 days.

The responses have caused global social and economic disruption, including the largest global recession since the Great Depression. It has led to the postponement or cancellation of events, widespread supply shortages exacerbated by panic buying, famines affecting hundreds of millions of people, and decreased emissions of pollutants and greenhouse gases. Educational institutions have been partially or fully closed. Misinformation has circulated through social media and mass media.

In response to these conditions, there has been a tremendous scientific effort to develop both prophylactic vaccines and medication treatments. Despite the development of several vaccines, and emerging treatment protocols, transmission and infection remains high, as the virus mutates into different forms (e.g., so-called Delta variant) evading current protocols. Thus, there remains a need in the art for new and effective treatments which inhibit the replication of SARS-CoV-2 virus, reduce viral transmission, and prevent or ameliorate symptoms of COVID-19. There is accordingly a need in the art for new and effective treatments which inhibit the replication of SARS-CoV-2 virus and prevent or ameliorate COVID-19.

### SUMMARY OF THE INVENTION

The present invention provides important formulations useful for inhibiting SARS-CoV-2 virus and in the treatment of COVID-19 disease. Generally, the formulations are mixtures of turmeric, Peganum harmala, and Arum palaestinum, or variants thereof, and which may also include additional minor ingredients. The methods involve the administration of the formulations to a subject in prophylactically or therapeutically effective amounts which inhibit replication of the SARS-CoV-2 virus in the subject.

The use of the formulations in the manufacture of a medicament for such treatment is also within the ambit of the invention.

The present disclosure also includes methods of inhibiting viral replication by contacting SARS-CoV-2 virus with the formulations in an effective amount and for an effective period of time in order to inhibit the growth, replication, and/or viability of the virus. Such usages would typically be in vitro.

### DETAILED DESCRIPTION OF THE PREFERRED EMBODIMENTS

Methods described herein include those for inhibiting replication of coronavirus, and prevention or amelioration of COVID-19. In one aspect, the methods comprise (consist essentially or even consist of) contacting coronavirus with amounts of at a least three-component formulation comprising (consisting essentially of, or even consisting of) turmeric, Peganum harmala, and Arum palaestinum, effective to inhibit viral replication for an effective period of time. In another aspect, the methods comprise (consist essentially or even consist of) administering a therapeutically or prophylactically effective amount of the formulations to human subjects in need thereof. The subjects may be at risk of viral infection or exposure to coronavirus and/or may be individuals identified as having been infected with coronavirus. Such individuals may be symptomatic or asymptomatic. Thus, “therapeutic” use of the formulation refers to processes that are intended to produce a beneficial change in an existing condition (e.g., viral infection) of the subjects, such as by reducing the severity of the clinical symptoms and/or effects of the infection, and/or reducing the duration of the infection/symptoms/effects. Likewise, “prophylactic” use of the formulation refers to processes that are intended to inhibit or ameliorate the effects of a future viral infection to which subjects may be exposed (but is not currently infected with). In some cases, the formulations may prevent the development of observable morbidity from viral infection (i.e., near 100% prevention). In other cases, the formulations may only partially prevent and/or lessen the extent of morbidity due to the viral infection (i.e., reduce the severity of the symptoms and/or effects of the infection, and/or reduce the duration of the infection/symptoms/effects). In either case, the formulations are still considered to “prevent” the target infection.

### The Formulations of the Invention

In their broadest aspects, the formulations used in the context of the invention comprise (or consist essentially of, or consist of) individual quantities of turmeric, Peganum harmala, and Arum palaestinum, as the principal ingredients working synergistically to provide health benefits.

Arum species, particularly Arum palaestinum, have a long history of diverse clinical applications, including the treatment of infections, bone fractures, and cancer. See, for example, Said et al. Ethnopharmacological Survey of Medicinal Herbs in Israel, the Golan Heights and the West Bank Region. J. Ethnopharmacology. 83 (2002): 251-265. In use, Arum plant parts, including roots, stems, and leaves, are often boiled in water to remove toxicity that may be present in the raw form of the plant, whereupon the plant parts are dried and ground, or formulated as liquids. The portfolio of chemical constituents in Arum palaestinum includes phytochemicals, such as flavonoids, phenolic acids and their derivatives, and turpenoid derivatives. See, U.S. Pat. No. 8,039,025, incorporated by reference herein.

Peganum harmala is a perennial, glabrous plant which grows spontaneously in the Eastern Mediterranean region. The plant is well known in Iran and is widely distributed and used in the support and maintenance of human health. Studies carried out on the chemical compositions of Peganum harmala extracts confirm that beta-carboline and quinazoline alkaloids are important compounds of this plant. The plant is one of the most frequently used plants to aide in hypertension and cardiac health worldwide. See, Moloudizargari et al, Pharmacological and therapeutic effects of Peganum harmala and its main alkaloids, Pharmacogn Rev. 7(14): pp. 199-212, 2013.

Curcuma longa (turmeric) has been used for thousands of years as a spice and medicinal herb. Turmeric includes minor amounts of curcumin, which has shown to provide anti-inflammatory effects and is a strong anti-oxidant. β-sitosterol is a plant sterol which offers a number of health benefits, whether it naturally occurs in foods or as a part of supplements. See, Cooper, Health Benefits of β-sitosterol, Livestrong.com, Aug. 14, 2017. Vitamin C and garlic also have well-known health benefits.

While the aforementioned products have individually shown promise for various aspects of human health, there have heretofore been no attempts to combine these ingredients for the preparation of beneficial nutritional supplements exhibiting synergistic effects in an antiviral capacity.

The present disclosure provides a synergistic antiviral formulation comprising individual quantities of turmeric, Peganum harmala, and Arum palaestinum, in a blended formulation which produce synergistic results in inhibiting coronavirus. As used herein, synergy or synergism means that the three-component compositions of the invention exhibit a joint action where the compositions produce an effect greater than that which may be obtained by use of individual components in equivalent quantities.

In such three-component formulations, the amount of turmeric is greater than either of Peganum harmala and Arum palaestinum, e.g., such three-component formulations would typically include from about 40-70% turmeric, and from about 15-35% each of Peganum harmala and Arum palaestinum. More preferred formulations include these three ingredients together with β-sitosterol, garlic, and Vitamin C, and again the amount of turmeric is preferably greater than any of these other ingredients. In one or more embodiments, the synergistic formulation comprises (consists essentially of, or even consists of) turmeric, Peganum harmala, Arum palaestinum, (3-sitosterol, garlic, and Vitamin C, effective to inhibit viral replication. For example, such six-component formulations would typically include from about 15-25% turmeric, from about 5-15% of Peganum harmala, from about 5-15% of Arum palaestinum, from about 5-15% of β-sitosterol, from about 5-15% of garlic, and from about 8-20% Vitamin C. Nutritional supplements comprising such synergistic formulations for promotion of normal blood glucose and cholesterol levels, for the promotion of prostate health, and for supporting normal cellular division are described in U.S. Pat. No. 10,772,347, issued Sep. 15, 2020, and entitled Nutritional Supplement, which is incorporated by reference in its entirety herein, and may be provided under the tradename “Afaya Plus.”

The formulations may also include significant amounts of vanillin compound(s), such as one or more of vanillin, vanilla, isovanillin, orthovanillin, and ethyl vanillin, which would correspondingly reduce the amounts of the other ingredients therein. In one or more embodiments, the synergistic formulation comprises (consists essentially of, or even consists of) turmeric, Peganum harmala, Arum palaestinum, β-sitosterol, garlic, vanillin compound(s), and Vitamin C, effective to inhibit viral replication. In certain embodiments, the formulations may contain from about 8-15% (more preferably from about 9-11%) Vitamin C, from about 5-11% (more preferably from about 7-9%) garlic, from about 35-45% (more preferably from about 38-42%) vanillin compound(s), from about 5-11% (more preferably from about 7-9%) β-sitosterol, from about 12-20% (more preferably from about 14-17) turmeric, from about 5-11% (more preferably from about 7-9%) Peganum harmala, and from about 5-11% (more preferably from about 7-9%) Arum palaestinum. Additional ingredients may be included in the production of complete formulations, including other active agents, preservatives, buffering agents, salts, carriers, excipients, diluents, or other ingredients.

All of the foregoing percentages are based on the total weight of the formulations taken as 100% by weight, including the three-component formulations and those containing additional other ingredients.

The ingredients of the formulations, and especially the turmeric ingredient, can be in the form of the above compounds per se, or alternately possible relevant variants thereof. Such variants include the isomers, tautomers, enantiomers, esters, derivatives, metal complexes (e.g., Cu, Fe, Zn, Pt, V), prodrugs, solvates, metabolites, and pharmaceutically acceptable salts of turmeric; such variants are generally not apropos to Peganum harmala and Arum palaestinum, save for solvates, inasmuch as these products are mixtures of many individual compounds.

“Isomers” refers to each of two or more compounds with the same formula but with at different arrangement of atoms, and includes structural isomers and stereoisomers (e.g., geometric isomers and enantiomers); “tautomers” refers to two or more isometric compounds that exist in equilibrium, such as keto-enol and imine and enamine tautomers; “derivatives” refers to compounds that can be imagined to arise or actually be synthesized from a defined parent compound by replacement of one atom with another atom or a group of atoms; “solvates” refers to interaction with a defined compound with a solvent to form a stabilized solute species; “metabolites” refers to a defined compound which has been metabolized in vivo by digestion or other bodily chemical processes; and “prodrugs” refers to defined compound which has been generated by a metabolic process. The compounds can be directly used in partial or essentially completely purified forms, or can be modified as indicated above. The compounds may be in crystalline or amorphous forms, and may be lyophilized.

Some or all of the ingredients can be in the form of acceptable esters or salts. The esters and salts should be generally safe, non-toxic, and neither biologically nor otherwise undesirable and are acceptable for human use, and which possess the desired degree of activity. Accordingly, the recitation herein of the ingredients is intended to embrace not only the named ingredients, but also any acceptable esters or salts thereof.

“Pharmaceutically acceptable salts” with reference to the components means salts of the components which are pharmaceutically acceptable, i.e., salts which are useful in preparing pharmaceutical compositions that are generally safe, non-toxic, and neither biologically nor otherwise undesirable and are acceptable for human pharmaceutical use, and which possess the desired degree of pharmacological activity. Such pharmaceutically acceptable salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts Properties, and Use, P. H. Stahl & C. G. Wermuth eds., ISBN 978-3-90639-058-1 (2008).

Advantageously, all of the ingredients are at least food grade and are blended together as powders. The Peganum harmala and Arum palaestinum powders may be obtained by drying and pulverizing the complete plants, including leaves, stems, and bulbs. In one or more embodiments, fresh plant parts (stems, leaves, bulbs, etc.) are macerated into a pulp and extracted with aqueous ethanol and/or water. In one or more embodiments, the macerated plant parts are first subjected to air drying before extraction. In one or more embodiments, the macerated plant parts are first baked, followed by grinding into a powder before extraction. In one or more embodiments, the complete plant parts are first dried and then pulverized into a powder, which can be used directly or first extracted. It will be appreciated that it is not essential that all of the plant parts be used, i.e., use may be made of the leaves, and/or stems, and/or bulbs. Regardless, the resulting extracts can then be suspended in aqueous ethanol, centrifuged, and filter sterilized to yield the resulting dosage forms. If desired, the cooled mixture can be lyophilized (freeze-dried) to obtain a dried extract. This extract may be then put in capsule form or may be tableted to provide solid dosage forms.

In one or more embodiments, in solid dosage form, the blended powders should be of a size to pass through a 50-mesh screen while being retained by a 100-mesh screen. Still further, the blended powders are placed in capsules for ease of dosage. The capsules or tablets should each contain from about 500-2000 mg of the blended powders, preferably from about 800 mg to about 1500 mg, most preferably about 1000 mg of the blended powders. While such powdered formulations are preferred for ease of manufacture and administration, it should be understood that the invention is not so limited. For example, the blended ingredients may be prepared as liquid dispersions or solutions using appropriate, non-interfering dispersants or solvents; other possible dosage forms include gels, suspensions, or solids such as tablets or pills.

### Use of the Formulations

In use, therapeutically effective amounts of the formulation are administered to a mammalian subject in need thereof for a therapeutically effective amount of time. As used herein, a “therapeutically effective” amount refers to the dosage amount and/or duration that will elicit the biological or medical response of a tissue, system, or subject that is being sought by a researcher or clinician, and in particular elicit some desired therapeutic effect as against the coronavirus cells by slowing and/or inhibiting activity, growth, or replication of the cells. One of skill in the art recognizes that an amount or duration may be considered therapeutically “effective” even if the condition is not totally eradicated or prevented, but it or its symptoms and/or effects are improved or alleviated partially or inhibited from worsening in the subject. Such therapeutically effective dosages and durations may comprise a single unit dosage or, more usually, periodic (e.g., daily or weekly) administration of lower dosages over time.

The formulations of the invention, in whatever physical form, are designed for administration to humans, in particular those exposed, or potentially exposed, to the SARS-CoV-2 virus, or those suffering from the COVID-19 disease. The formulations may be administered in any convenient manner, such as by oral, rectal, nasal, ophthalmic, parenteral (including intraperitoneal, gastrointestinal, intrathecal, intravenous, cutaneous (e.g., dermal patch), subcutaneous (e.g., injection or implant), or intramuscular) routes. In one or more embodiments, the dosage formulations are administered in repeated dosages, e.g., daily, multiple time daily, and the like. Where the aforementioned capsules are used, the administration would be oral, and the recommended dosage level would be four such capsules per day, taken twice daily, two capsules per serving. Dosage forms may be administered with a meal, or suspended or dissolved in PEPTAMEN® or other fat-containing liquid before administration, or co-administered with such fatty substances. A clinician or researcher may determine the appropriate administration protocol depending upon the dosage form and route of administration used.

The formulation may be administered in conjunction with other recommended therapies for treating COVID-19 or its symptoms. The formulation may also be taken prophylactically, such as during period of time where there is high community transmission of SARS-CoV-2. The methods can be also applied for clinical research and/or study.

Additional advantages of the various embodiments of the invention will be apparent to those skilled in the art upon review of the disclosure herein. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

Levels of dosing to human subjects of the formulations hereof are quite variable owing to factors such as the patient's age, patient's physical condition, and the severity of the disease. In general, however, regardless of the dosage form or route of administration employed, the formulations should be dosed of from about 5 to 2000 mg per day, and more usually from about 100-800 mg per day. Such dosages may be based on a single administration per day, but more usually multiple administrations per day.

As used herein, the term “inhibit” refers to a reduction or decrease viral titer or quantity, compared to a baseline. For example, in the context of the present invention, inhibition of viral replication refers to a decrease in amount or speed of viral replication as compared to baseline (e.g., as detected by a rapid antigen test, or molecular/PCR test, or other suitable testing methodology). By comparing a baseline obtained before administration of the formulation to the values obtained from the individual after administration of the formulation, those of ordinary skill in the art can readily determine whether or not viral replication has been inhibited and to what extent. Thus, an “effective amount” to inhibit viral replication refers to the amount of a given formulation that results in a reduced level of viral replication and thus a reduced amount of detectable virus in the individual (e.g., reduced viral titer or viral load) when comparing the baseline detected amount to the reduced amount using the same testing methodology. Preferably, prophylactic and/or therapeutic methods of the invention will lead to a decrease of at least 20%, at least 30%, at least 40%, at least 50%, at least 60%, at least 70%, at least 80%, or even at least 90% as compared to the baseline. Preferably, such reductions are seen within 72 hours after administration, preferably within 48 hours, preferably within 36 hours, more preferably within 24 hours. Correspondingly, such reductions in viral load will advantageously lead to an amelioration, improvement, or decrease in one or more symptoms associated with coronavirus infection and/or reduced transmission of the virus from the infected individual. Alternatively, inhibition or antiviral efficacy can be assessed in vitro. In one or more embodiments, the formulation will inhibit viral replication by at least 50% in a cell-based assay, preferably by at least 60%, 70%, 80%, or 90% in a cell-based assay. Preferably, the formulation will have a Selectivity Index of greater than 1, preferable of at least 3, more preferably at least 5, and more preferably at least 6. The Selectivity Index is the ratio of the toxic concentration of a formulation against its effective bioactive amount. In one or more embodiments, the toxic concentration is the dose that leads to 50% cell cytotoxicity in an in vitro cell assay. In one or more embodiments, the bioactive amount is the dose that inhibits viral replication by 90% in a cell assay.

Furthermore, the phrase “and/or,” when used in a list of two or more items, means that any one of the listed items can be employed by itself or any combination of two or more of the listed items can be employed. For example, if a formulation is described as containing or excluding components A, B, and/or C, the formulation can contain or exclude A alone; B alone; C alone; A and B in combination; A and C in combination; B and C in combination; or A, B, and C in combination.

### Examples

The following example sets forth methods in accordance with the invention. It is to be understood, however, that this example is provided by way of illustration and nothing therein should be taken as a limitation upon the overall scope of the invention.

Formulation Analysis and Preparation

### Materials and Methods.

Arum palaestinum Boiss were identified morphologically during field collection from growing habitats in the Palestinian region, and seeds from the plants were then planted in a growth chamber for germination (samples are on file at the Missouri Botanical Garden (MBG) for verification, voucher #Croat 95,466 (MO)). The plant extract was analyzed using Gas Chromatography-Mass Spectroscopy (GC-MS).

GCMS RESULTS

| | | Relative |
|---|---|---|
| Molecule | Formula | Amount |
| hexadecanoic acid | C16H32O2 | 1 |
| linolenic acid | C18H30O2 | 0.48 |
| linoleic acid | C18H32O2 | 0.44 |
| oleamide | C18H35NO | 0.12 |
| 2-monopalmitin | C19H38O4 | 0.17 |
| phytol | C20H40O | 0.49 |
| campesterol | C28H48O | 0.79 |
| sitostenone | C29H48O | 0.085 |
| stigmasterol | C29H48O | 0.29 |
| isofucosterol | C29H48O | 0.13 |
| 5a-stigmastane-3,6-dione | C29H48O2 | 0.038 |
| beta-sitosterol | C29H50O | 1.9 |
| cycloartenol | C30H50O | 0.19 |
| dl-a-tocopherol | C29H50O2 | 0.050 |
| heneicosane | C21H44 | 0.028 |
| tricosane | C23H48 | 0.093 |
| pentacosane | C25H52 | 0.29 |
| heptacosane | C27H56 | 0.88 |
| nonacosane | C29H60 | 2.1 |
| hentriacontane | C31H64 | 0.39 |
| 5,5,8a-Trimethyl-3,5,6,7,8,8a- | C12H20O | 0.15 |
| hexahydro-2H-chromene | | |
| 6-(3,3-Dimethyl-oxiran-2-ylidene)-5,5- | C12H18O2 | 0.029 |
| dimethyl-hex-3-en-2-one | | |
| 2-butanone, 4(2,6,6-trimethyl-1,3 | C13H20O | 0.070 |
| cyclohexadien-1-yl) | | |
| 2-cyclohexen-1-one, 4-(3-hydroxy-1- | C13H20O2 | 0.042 |
| butenyl)-3,5,5-trimethyl | | |
| 2-cyclohexen-1-one, 4-(3-hydroxybutyl)- | C13H22O2 | 0.12 |
| 3,5,5-trimethyl- | | |
| 6-(3-Hydroxy-but-1-enyl)-1,5,5- | C13H22O3 | 0.17 |
| trimethyl-7-oxabicyclo[4.1.0]heptan-2-ol | | |
| 3-Buten-2-one, 4-(4-hydroxy-2,2,6- | C13H20O3 | 0.05 |
| trimethyl-7-oxabicyclo[4.1.0]hept-1-yl)- | | |
| isovanillin | C8H8O3 | 0.012 |
| cinnamic acid | C9H8O2 | 0.018 |
| 2 methoxy 4 vinylphenol | C9H10O2 | 0.035 |
| 2-propenal, 3-(4-hydroxy-3- | C10H10O3 | 0.063 |
| methoxyphenyl) | | |
| docosyl hexadecanate | C16H33O2-C22H44 | 0.081 |

Portions of the plants were cut into wedges and boiled in water for 4 hours, whereupon the wedges were dried to completion in an 80° C. oven and ground into powder for use. Peganum harmala seeds and powdered Curcuma longa (turmeric) root were purchased from commercial sources.

For capsule dosage forms 106 mg of vitamin C powder is mixed with 894 mg of a proprietary blend of vanillin powder, turmeric powder, harmala powder, garlic powder, Arum Palaestinum powder, and beta-sitosterol powder in the described ratios. The mixture can be formulated into gelatin capsules.

In Vitro Assay of COVID 19 Using Synergistic Formulation

The antiviral activity of a synergistic formulation on coronavirus was assessed in vitro using novel coronavirus SARS-CoV-2 isolate USA-WA1/2020 (deposited by the Centers for Disease Control and Prevention). For propagation and experimentation with SARS-CoV-2, we used Vero E6 (ATCC® CRL-1586™) cells maintained in EMEM (Eagle's Minimum Essential Medium) cell culture media (cat #30-2003, ATCC, Manassas, Va., USA) supplemented with 2% or 10% fetal bovine serum (FBS), 100 U/mL penicillin, 100 μg/mL streptomycin, 0.01M HEPES buffer solution, 1 mM sodium pyruvate, 1× non-essential amino acids solution (cat #SH3023801, Thermo Fisher Scientific, Waltham, Mass., USA), and 2 mM L-glutamine.

The synergist formulation containing a blend of turmeric, Peganum harmala, Arum palaestinum, β-sitosterol, garlic, and Vitamin C powder was tested. The powder blend was suspended in aqueous ethanol in a 1:10 wt:volume ratio, centrifuged and filter sterilized. For comparison, testing was also carried out using only Arum palaestinum extract prepared from fresh Arum bulb, macerated into a pulp and extracted with 70% EtOH. The resulting extract was suspended in aqueous ethanol in a 1:10 wt:volume ratio, centrifuged and filter sterilized.

Plated cells were first infected with SARS-CoV2 at a multiplicity of infection (MOI) of 0.001. The test formulations were added during and post-infection. At 72 hours post-infection, the cell monolayers were stained with crystal violet to determine the level of cytopathic effect (CPE). The CPE level was quantified to determine the IC₉₀ (dose that inhibited viral replication by 90%). Vero cells were also treated with the test formulations alone for 24 hours to determine the CC₅₀ (dose that leads to 50% cell cytotoxicity). After 24 hours of treatment, cell viability was determined by a standard MTS colorimetric assay. Based on the IC₉₀ and CC₅₀, the Selectivity Index (CC₅₀/IC₉₀) of each of the treatments could be determined. A higher Selectivity Index indicates that the treatment will, theoretically, be both safer and more effective during in vivo treatment for a given viral infection. The results are in the table below.

TABLE

| Test Formulation | CC₅₀(μl/ml) | IC₉₀(μl/ml) | Selectivity Index |
|---|---|---|---|
| Synergistic blend | 1.2 | <0.1-0.2 | ~6-12 |
| A. palaestiunum, | 55 | 16 | 3.4 |
| 70% EtOH, fresh | | | |

As can be seen, the synergistic blend has a low toxicity profile, while also showing antiviral activity at very low dosages. Overall, it presents a higher selectivity index as compared to the Arum palaestinum extract alone, indicating it would be effective against SARS-CoV-2 infection at low dosages, as well as safe to administer.

1. A method of inhibiting viral replication in a human subject exposed to or at risk of exposure to SARS-CoV-2 virus, or suffering from COVID-19, comprising the step of administering to the subject a prophylactically or therapeutically effective amount of a formulation comprising a synergistic blend of turmeric, Peganum harmala, and Arum palaestinum, and/or the relevant isomers, tautomers, enantiomers, esters, derivatives, metal complexes, prodrugs, solvates, metabolites, and pharmaceutically acceptable salts of the foregoing, which said formulation inhibits replication of said SARS-CoV-2 virus in said subject.

2. The method of claim 1, said formulation further comprising one or more of vitamin C, vanillin, garlic, and/or beta-sitosterol.

3. The method of claim 1, said formulation being administered to said human subject by a route selected from the group consisting of oral, rectal, nasal, ophthalmic, and parenteral administrations.

4. The method of claim 1, said formulation being administered at a level of from about 5 to 2000 mg per day.

5. The method of claim 1, wherein said human subject is suffering from one or more symptoms of COVID-19 prior to said administration step.

6. The method of claim 1, wherein said human subject is asymptomatic for COVID-19 prior to said administration step.

7. The method of claim 1, wherein said human subject has a baseline amount of detectable SARS-CoV-2 virus prior to said administration step, wherein after said administration step, said baseline amount of detectable virus is reduced.

8. The method of claim 7, wherein the baseline amount of detectable virus is reduced by at least 20% within 72 hours after said administration step.

9. A pharmaceutical formulation comprising a synergistic blend of turmeric, Peganum harmala, and Arum palaestinum, and/or the relevant isomers, tautomers, enantiomers, esters, derivatives, metal complexes, prodrugs, solvates, metabolites, and pharmaceutically acceptable salts of the foregoing, for inhibiting replication of SARS-CoV-2 virus in a subject or for the treatment of a human subject suffering from COVID-19.

10. The pharmaceutical formulation of claim 9, said formulation being in a solid dosage form comprising said synergistic blend of turmeric, Peganum harmala, and Arum palaestinum powder in a capsule.

11. The pharmaceutical formulation of claim 9, said formulation further comprising one or more of vitamin C, vanillin, garlic, and/or beta-sitosterol.

12. A method of inhibiting SARS-CoV-2 viral replication, comprising the step of contacting said virus with a formulation comprising turmeric, Peganum harmala, and Arum palaestinum, and/or relevant the isomers, tautomers, enantiomers, esters, derivatives, metal complexes, prodrugs, solvates, metabolites, and pharmaceutically acceptable salts of the foregoing.
